(19) United States
(12) Patent Application Publication
(10) Pub. No.: US 2026/0208086 A1
(43) Pub. Date: Jul. 23, 2026

(54) SYSTEMS AND METHODS FOR SCHEDULING FILTER MAINTENANCE OF AN IMAGING SYSTEM

(71) Applicant: GE Precision Healthcare LLC, Waukesha, WI (US)

(72) Inventors: Chad Smith, Franklin, WI (US); Michelle DeLong Samalik, Franklin, WI (US); Brandon Smith, Waukesha, WI (US); Donald Murray, West Bend, WI (US); Sam Maule, Hartford, WI (US); Joseph Maule, Menomonee Falls, WI (US); Anand Amirtharaj Amirtharaj Arokiaswamy, Waukesha, WI (US); Chelsey Lewis, Brookfield, WI (US); Phil Huettl, Waukesha, WI (US)

(21) Appl. No.: 19/035,373

(22) Filed: Jan. 23, 2025

(51) Int. Cl. B01D 46/00; B01D 46/18; B01D 46/681; B01D 46/682; G01N 7/00; G21K 1/10

(52) U.S. Cl. CPC B01D 46/0086; B01D 46/18; B01D 46/681; B01D 46/682; G01N 7/00; G21K 1/10; B01D 2273/28; B01D 2279/45

(57) ABSTRACT

Various systems and methods for scheduling filter maintenance for an imaging system are described herein. An example method for scheduling filter maintenance for an imaging system includes detecting, via a sensor, a pressure differential across a filter is below a threshold, providing an alert to an operator, via a display of the imaging system, that the pressure differential across the filter is below the threshold, and scheduling filter maintenance based on the pressure differential being below the threshold.

### BACKGROUND

Embodiments of the subject matter disclosed herein relate to systems and methods for determining when gantry air filters of a computed tomography (CT) imaging system need to be cleaned to improve temperature regulation and operating efficiency of the CT imaging system. In some embodiments, upon determining gantry air filters of the CT imaging system need to be cleaned, a filter cleaning device coupled to the CT imaging system is initiated.

Imaging systems, such as computed tomography (CT) imaging systems include components, such as detectors, X-ray generators, and processors, which generate heat during operation. To cool off the system, cooler air (e.g.., from a surrounding environment) may be drawn in through an inlet or inlets in the gantry via one or more fans and circulated through the gantry. At least one filter is positioned adjacent the inlet to prevent dust and other particulate in the air from entering the gantry interfering with operation of the gantry. As the filter(s) trap more dust and particulate, the filters allow less air to pass through to the inlet, thus reducing the cooling capability. To ensure the flow rate through the filters remains sufficient for cooling the components of the gantry, the filter(s) are cleaned and/or replaced during regular maintenance intervals. However, in some environments, the filters may need cleaning more often or between regularly scheduled maintenance appointments, and in other environments, less cleaning may be required so time between maintenance appointments can be longer.

### SUMMARY

This summary introduces concepts that are described in more detail in the detailed description. It should not be used to identify essential features of the claimed subject matter, nor to limit the scope of the claimed subject matter.

In an aspect described herein, an example method for scheduling filter maintenance for an imaging system includes detecting, via a sensor, a pressure differential across a filter is above a threshold, providing an alert to an operator, via a display of the imaging system, that the pressure differential across the filter is above the threshold, and scheduling filter maintenance based on the pressure differential being below the threshold.

In another aspect described herein, an example imaging system includes a housing, an inlet for air positioned within the housing, a filter positioned adjacent to the inlet, a display including a user interface, one or more sensors adjacent to the filter, the one or more sensors to measure a pressure differential across the filter, and a processor. The processor includes instructions to determine the pressure differential is above a threshold, provide an alert to an operator via the user interface of the display that the pressure differential is above the threshold, and schedule filter maintenance based on pressure differential being below the threshold.

### BRIEF DESCRIPTION OF DRAWINGS

The accompanying drawings included in the present application are intended to help to further understand embodiments of the present application, constitute a part of the specification, and are used to illustrate implementations of the present application and set forth the principles of the present application together with textual description. Obviously, the accompanying drawings in the following description are merely some embodiments of the present application, and a person of ordinary skill in the art could obtain other implementations according to the accompanying drawings without the exercise of inventive effort. In the accompanying drawings:

FIG. 1 shows a perspective view of an imaging system according to an embodiment of the present disclosure.

FIG. 2 shows a block diagram of an imaging system according to an embodiment of the present disclosure.

FIG. 3 depicts an example gantry of the imaging system of FIGS. 1 and 2 including pressure sensors positioned adjacent to filters at air inlets.

FIG. 4 depicts a first example of a filter cleaning device for the filter at the inlet of the gantry.

FIG. 5 depicts a second example of a filter cleaning device for the filter at the inlet of the gantry.

FIG. 6 depicts a third example of a filter cleaning device for the filter at the inlet of the gantry.

FIG. 7 depicts a fourth example of a filter cleaning device for the filter at the inlet of the gantry.

FIG. 8 depicts a flowchart representing a method of scheduling filter maintenance.

FIG. 9 depicts a flowchart representing another method of scheduling filter maintenance.

FIG. 10 depicts a flowchart representing an additional method of scheduling filter maintenance.

FIG. 11 depicts example alerts that may be provided to the user indicating filter maintenance is required.

### DETAILED DESCRIPTION

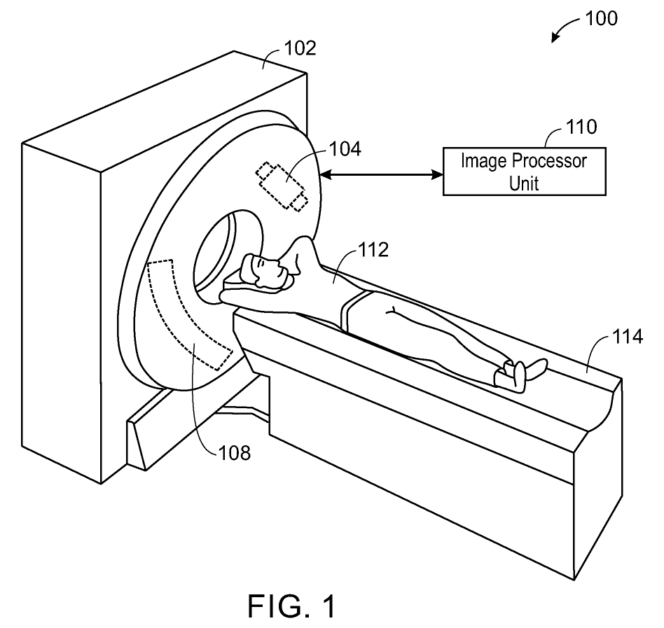

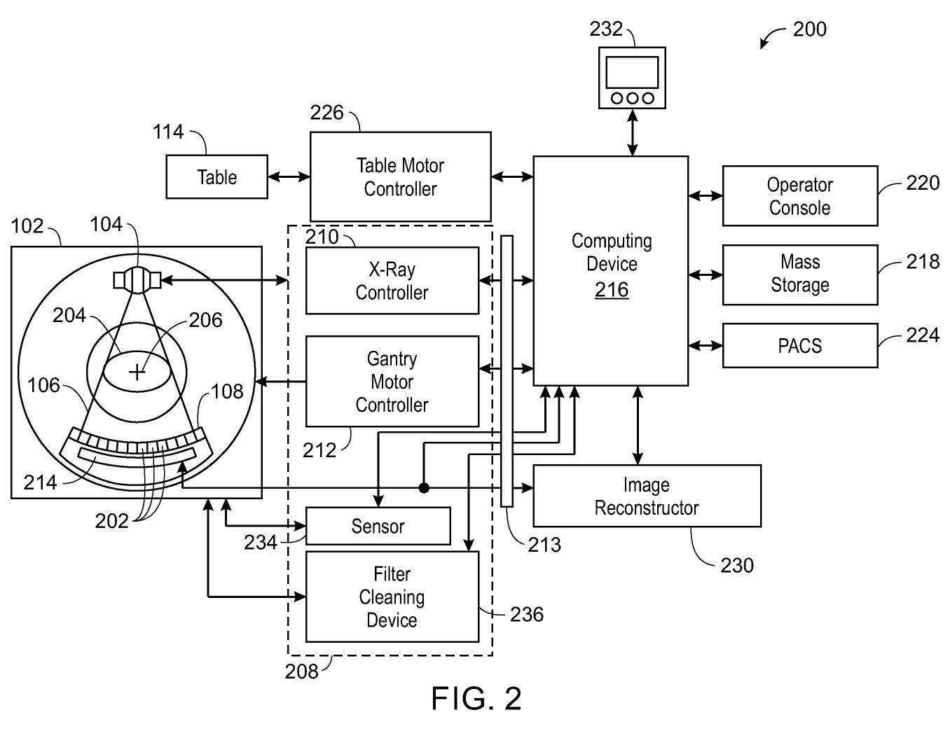

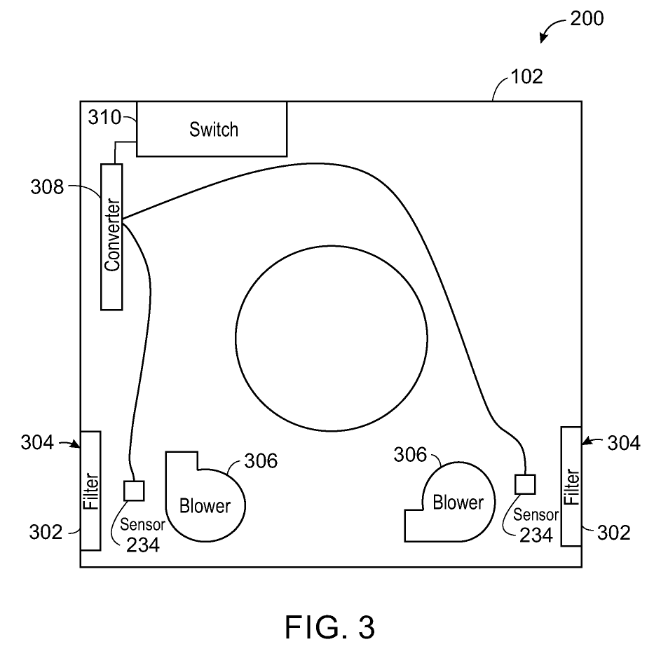

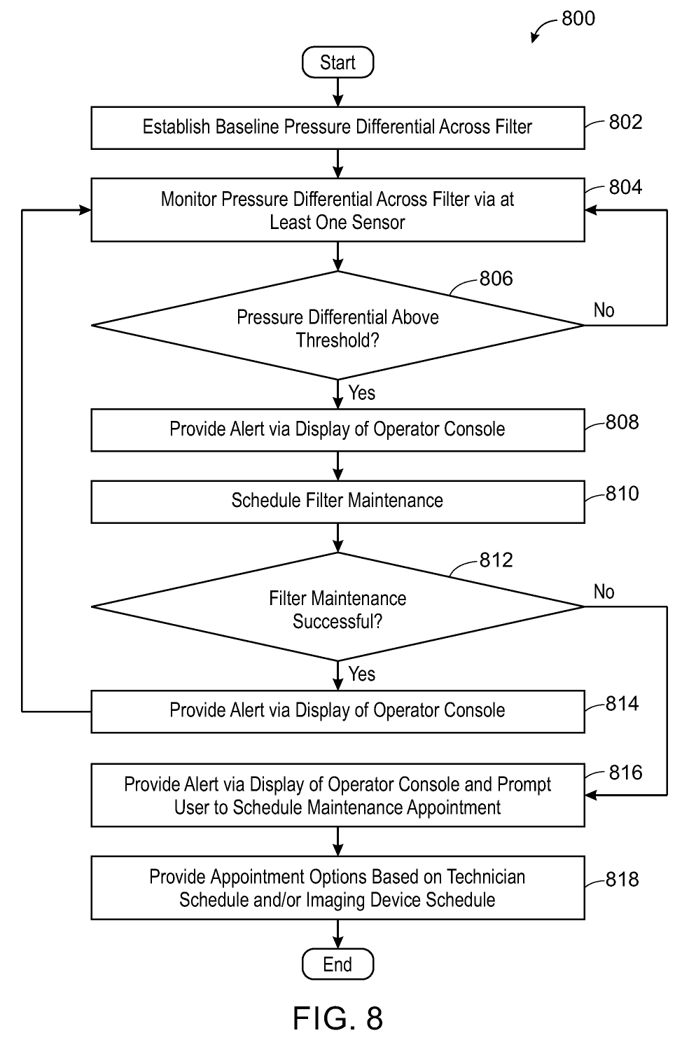

Embodiments of the present disclosure will now be described, by way of example, with reference to the Figures, in which FIGS. 1 and 2 depict an example imaging system (e.g., a Computed Tomography (CT) imaging system) with which the example systems, apparatus, and methods described herein may be used. Alternatively, other types of imaging systems may be used. FIGS. 3-7 depict various example systems an apparatus for scheduling filter maintenance of an imaging system, as described herein. FIGS. 8-10 depict various example flowcharts representing methods for scheduling maintenance of an imaging system, as described herein.

In particular, the example systems and methods described herein for use with an imaging system, such as that depicted in FIGS. 1 and 2, includes at least one sensor (e.g., one or more sensors) positioned adjacent a filter of an inlet of a housing (e.g., a gantry housing) of an imaging system. The sensor(s) monitor a pressure differential over the filter (e.g., how much an air pressure changes before and after the filter). The measured pressure differential indicates how much dirt is on the filter (e.g., how clogged the filter is, how dirty the filter is, etc.). A filter with too much dirt prevents sufficient air flow for cooling components of the imaging system. The example apparatus and systems described herein may also include one or more filter cleaning devices. Filter cleaning devices may include vacuums, brushes/bristles, adhesive or sticky sheets, blowers, additional filter material, or a combination thereof. In some examples, when a computing device determines the filter has accumulated too much dirt (e.g., based on a pressure differential measured by the sensors), the computing device sends a signal to the filter cleaning device to activate and clean the filter. Additionally, and/or alternatively, the computing device provides an alert to an operator to schedule maintenance and/or schedules maintenance automatically. In this way, it can be ensured that the filter of the imaging system is sufficiently clean to allow enough air through the filter to cool the imaging system, even between regularly scheduled preventative maintenance appointments.

FIG. 1 illustrates an exemplary imaging system 100. The illustrated imaging system 100 of FIG. 1 may be a Computed Tomography (CT) system. However, other types of imaging systems may be used with the methods, apparatus, and systems described herein, including but not limited to Photon Counting Computed Tomography (PCCT) systems, Magnetic Resonance Imaging (MRI) systems, Positron Emission Tomography (PET) systems, Single-Photon Emission Computed Tomography (SPECT) systems, and/or a combination of imaging systems. Particularly, the imaging system 100 is configured to image a subject 112 such as a patient, an inanimate object, one or more manufactured parts, and/or foreign objects such as dental implants, stents, and/or contrast agents present within the body. The imaging system 100 of FIG. 1 includes a gantry 102, which in turn, may further include at least one X-ray source 104 configured to project a beam of X-ray radiation 106 (see FIG. 2) for use in imaging the subject 112 laying on a table 114. Specifically, the X-ray source 104 is configured to project the X-ray radiation beams 106 towards a detector array 108 positioned on the opposite side of the gantry 102. Although FIG. 1 depicts a single X-ray source 104, in certain embodiments, multiple X-ray sources and detectors may be employed to project a plurality of X-ray radiation beams for acquiring projection data at the same or different energy levels corresponding to the patient. In some embodiments, the X-ray source 104 may enable dual-energy spectral imaging by rapid peak kilovoltage (kVp) switching. In the embodiments described herein, the X-ray detector employed is a photon counting detector which is capable of differentiating X-ray photons of different energies.

In certain embodiments, the imaging system 100 further includes an image processor unit 110 configured to reconstruct images of a target volume of the subject 112 using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. In some examples the image processor unit 110 may use an analytic image reconstruction approach such as FBP in addition to an iterative image reconstruction approach.

In some imaging system configurations, such as CT imaging systems, an X-ray source projects a cone-shaped X-ray radiation beam which is defined with respect to an X-Y-Z Cartesian coordinate system and generally referred to as an "imaging volume." The X-ray radiation beam passes through an object being imaged, such as the patient or subject. The X-ray radiation beam, after being attenuated by the object, impinges upon an array of detector elements. The intensity of the attenuated X-ray radiation beam received at the detector array is dependent upon the attenuation of an X-ray radiation beam by the object. Each detector element of the array produces a separate electrical signal that is a measurement of the X-ray beam attenuation at the detector location. The attenuation measurements from all the detector elements are acquired separately to produce a transmission profile.

In some CT systems, the X-ray source and the detector array are rotated with a gantry within the imaging volume and around the object to be imaged such that an angle at which the X-ray beam intersects the object constantly changes. A group of X-ray radiation attenuation measurements, e.g., projection data, from the detector array at one gantry angle is referred to as a "view." A "scan" of the object includes a set of views made at different gantry angles, or view angles, during one revolution of the X-ray source and detector.

FIG. 2 illustrates an exemplary imaging system 200 similar to the imaging system 100 of FIG. 1. As with FIG. 1, FIG. 2 depicts an imaging system that is a CT imaging system, but the apparatus and methods described herein may be used with a variety of other imaging systems. In accordance with aspects of the present disclosure, the imaging system 200 is configured for imaging a subject 204 (e.g., a patient, the subject 112 of FIG. 1). In one embodiment, the imaging system 200 includes the detector array 108 (see FIG. 1). The detector array 108 further includes a plurality of detector elements 202 that together sense the X-ray radiation beam 106 (see FIG. 2) that passes through the subject 204 (such as a patient) to acquire corresponding projection data. In some embodiments, the detector array 108 may be fabricated in a multi-slice configuration including the plurality of rows of cells or detector elements 202, where one or more additional rows of the detector elements 202 are arranged in a parallel configuration for acquiring the projection data. The detector elements 202 may also be referred to as pixels or detector pixels.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where the projection angle relative to the subject 204 varies as a function of time, the mounted components may be configured to move along a general curve rather than along a segment of a circle.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 204. The processed data are commonly called projections. In some examples, the individual detectors or detector elements 202 of the detector array 108 may include photon counting detectors which register the interactions of individual photons into one or more energy bins.

The acquired sets of projection data may be used for basis material decomposition (BMD). During BMD, the measured projections are converted to a set of material-density projections. The material-density projections may be reconstructed to form a set of material-density maps or images of each respective basis material, such as bone, soft tissue, and/or contrast agent maps. The density maps or images may be, in turn, associated to form a 3D volumetric image of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate data from a subset of the detector elements 202 into so-called macro-detectors. The data sampled and digitized by the DAS 214 is transmitted to a computer or computing device 216 via a slip ring 213. In one example, the computing device 216 stores the data in a storage device or mass storage 218. The storage device 218, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the X-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized X-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a "cloud" network cluster for the image reconstructor 230.

In one embodiment, the image reconstructor 230 stores the images reconstructed in the storage device 218. Alternatively, the image reconstructor 230 may transmit the reconstructed images to the computing device 216 to generate useful patient information for diagnosis and evaluation. In certain embodiments, the computing device 216 may transmit the reconstructed images and/or the patient information to a display or display device 232 having a user interface and communicatively coupled to the computing device 216 and/or the image reconstructor 230. In some embodiments, the reconstructed images may be transmitted from the computing device 216 or the image reconstructor 230 to the storage device 218 for short-term or long-term storage.

Information may be transmitted between the components residing in the gantry 102 and external devices (such as the computing device 216 and/or image reconstructor 230) via the slip ring 213, which facilitates electronic communication across the rotating gantry. In some examples, the gantry and internal components (e.g., the control mechanism 208, X-ray source 104, the detector array 108) may be collectively defined as a PCCT scanner, and as such the computing device 216 and image reconstructor 230 may reside off the scanner.

The example imaging system 200 includes at least one sensor 234. The example sensor 234 may include one or more sensor. In some examples, the sensor includes one or more flow rate sensors (e.g., a mass flow rate sensor, an air flow rate sensor, etc.) to measure a flow rate of air adjacent an inlet air filter (e.g., filter 302 of FIG. 3) of the housing or gantry housing of the imaging system. In some examples the sensor includes two flow rate sensors. In such examples, a first flow rate sensor is to measure a flow rate of the air at a point on one side of the filter (e.g., prior to the filter) and a second flow rate sensor is to measure a flow rate of the air at a point on the other side of the filter (e.g., after the filter). In this way, a difference in the flow rate measured before and after the filter may indicate a pressure differential across the filter. In other examples, a one or more differential pressure transducers may be positioned adjacent the filter to measure the pressure differential across the filter. A increase in the pressure differential across the filter may indicate a level of dirt accumulated by the filter.

In other examples, one or more pressure sensors may be positioned adjacent the filter to determine a pressure applied to the filter by the air passing through the filter. An increase in pressure may indicate more dirt is built up on or accumulated by the filter. In other examples, other sensors capable of measuring a pressure differential or otherwise determining the air filter has a build-up of dirt may be used. In some examples, other suitable air flow characteristic measuring devices may be used, including other sensors, meters, or valves. For example, an optical sensor to measure an amount of light passing through a filter. As dirt builds up on the sensor, less light passes through the filter.

In some examples, based on a signal from the senor 234, the computing device 216 determines if the measured value or a value determined based on the measurements of the sensor (e.g., pressure, pressure differential, optical change, etc.) meets a threshold. If the measured or determined value meets a threshold, the computing device may provide an alert to the operator via the display, for example, using the user interface. In some examples, the computing device 216 may alternatively or additionally automatically schedule a cleaning. Scheduling a cleaning may include determining when the imaging system is not being used (e.g., for an imaging scan) and scheduling a run time for the filter cleaning device. Alternatively or additionally, scheduling a cleaning may include scheduling a time for a technician to clean the filter based on the technician’s availability and the schedule of the imaging system (e.g., when the imaging system is scheduled for imaging scans). In some examples, scheduling a cleaning may include creating a task to be added to the next scheduled maintenance, either a regularly scheduled appointment for preventative maintenance and/or the next scheduled appointment for unexpected maintenance (e.g., the task is stored and added once the appointment is scheduled, but no additional appointment is scheduled). The example computing device 216 may track the cleaning tasks and determine whether the filter was cleaned successfully and when the filter was last cleaned and/or changed. In examples where a technician cleans the filter, the technician may provide a confirmation that the filter was cleaned or changed.

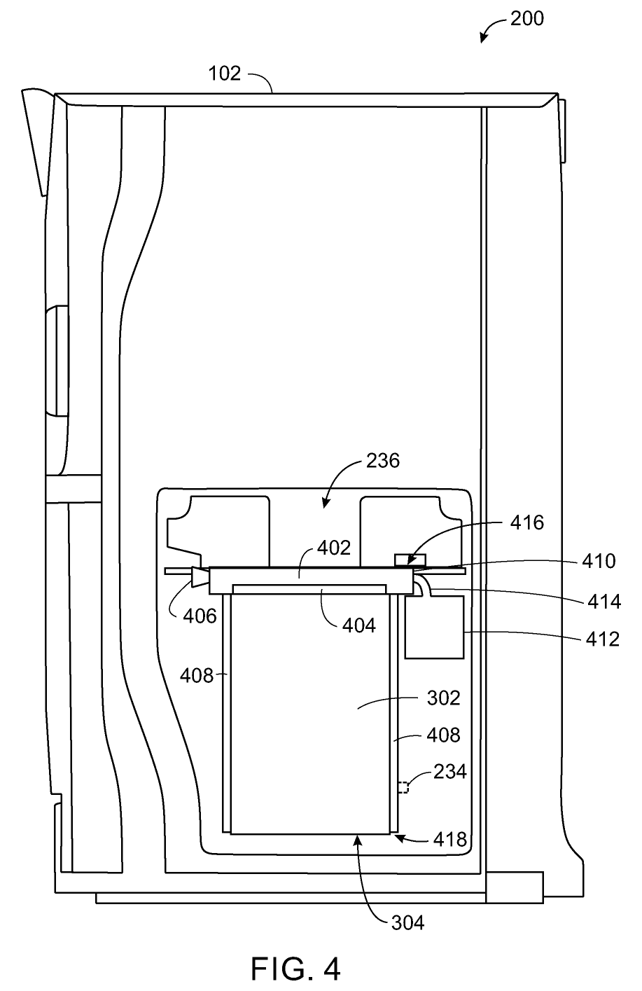

The example imaging system 200 may include a filter cleaning device 236. Filter cleaning devices 236 may include vacuums, brushes/bristles, adhesive or sticky sheets, blowers, additional filter material, or a combination thereof. In some examples, when a computing device determines the filter has accumulated too much dirt (e.g., based on a pressure differential measured by the sensors 234), the computing device 216 sends a signal to the filter cleaning device 236 to activate and clean the filter. The example filter cleaning device may perform the filter cleaning task at a time scheduled by the computing device. Example embodiments of filter cleaning devices may be described in more detail in conjunction with FIGS. 4-7. In some examples, one or more of the example filter cleaning devices and/or portions of the example cleaning devices may be combined and/or rearranged.

FIG. 3 depicts an example gantry 102 of an imaging system 200. FIG. 3 is a simplified schematic diagram of the gantry 102, depicting filters 302 positioned over inlets 304. The example sensors 234 (e.g., pressure sensors) are positioned adjacent to each filter. The inlet 304 may be an air inlet operative to intake air for cooling one or more components of the gantry, including the detector 108, the X-ray source 104, and/or any computing devices positioned within the gantry. Air may be drawn into and circulated through the gantry via one or more blowers 306 or fans. In some examples, the blowers 306 are positioned behind the respective inlets 304 to pull air in through the inlet 304. The example filters 302 may be a HEPA filter to prevent dirt (e.g., dust, particulate, etc.) from entering the gantry via the inlet 304, thereby preventing buildup of dirt within the gantry 102 that could interfere with the function of the imaging system 200. Although one filter 302 and inlet 304 are depicted, multiple filters 302 and inlets 304 may be included in the example gantry 102. Additionally, placement of the filter 302 and inlet 304 may be in any location on the gantry at which an inlet 304 may be needed to accommodate cooling of components of the imaging system 200, or at which an inlet may be positioned based on requirements of positioning for other components of the gantry 102. As described herein, the sensor 234 may be positioned adjacent to the filter and is operative to detect when the filter needs to be cleaned. Ideally, the sensors 234 are positioned between the filter 302 and the blower 306.

The pressure sensors 234 are communicatively coupled (e.g., via a wired connection, via a wireless connection) to a convertor 308 (e.g., a serial convertor). The convertor 308 converts the signal from the pressure sensors into a pressure differential value. The convertor 308 is further connected to a switch 310 (e.g., an ethernet switch) or other communication device. The switch 310 is communicatively coupled to the computing device 216. In some examples, the pressure sensor 234 and or a computing device 216 are operative to determine an altitude of the imaging system 200, which may affect the value of the pressure differential determined by the signal from the sensors 234. Alternatively, the altitude is provided manually by an operator at a time of setup for the imaging system. The sensors 234 may determine a baseline measurement or pressure differential at a known system configuration (e.g., during calibration). Additionally or alternatively, the sensors 234 may update the baseline measurement when the filter is replaced. A pressure differential threshold is defined based on the baseline measurement and the altitude of the imaging system. That is, if the pressure differential is below the pressure differential threshold, the filter 302 may need cleaned or replaced. In some examples, the pressure differential threshold is defined in a look-up table. In other examples, the pressure differential threshold is defined as a percentage of a baseline pressure differential measurement.

In some examples, a different type of sensor may be used, which measures a different value to determine when the filter needs cleaning or needs replaced. In such examples, a baseline value is determined during a calibration or filter installation. A threshold value may then be determined as a percentage over or under the baseline value. For example, if an optical sensor is used to determine an amount of dirt on the filter, a reduction in an amount of light passing through the filter that exceeds a predefined percentage of the baseline amount of light measured would be out of range (e.g., above a threshold, below a threshold), and would indicate filter cleaning is needed.

In some examples, the example pressure sensor 234 and computing device 216 provide a system score card to an operator or technician, which may include a pressure differential across the filters 302. The operator or technician is notified if a filter needs to be replaced or cleaned prior to a regularly scheduled preventative maintenance appointment. Additionally, the measured pressure differential can be recorded over time, and the computing device 216 can analyze trends for each filter 302 of the imaging system 200, even predicting when the filter 302 will need to be replaced or cleaned. Based on the trend data, pressure differential limits or thresholds can be established for each filter 302. The pressure limits can be a fixed value or can be expressed transfer function that uses variables sch as temperature, system duty cycle, site elevation, etc., which can more accurately determine when a filter needs to be cleaned or replaced. The computing device 216 can notify the user of the trends and alter the operator when the filter needs to be cleaned or replaced, particularly if cleaning or replacement is required between scheduled preventative maintenance. In some examples, predictive modeling can be used, based on the pressure differential data collected by the sensors 234, to estimate remining time until the pressure differential is below a certain threshold, allowing proactive scheduling for service.

While FIG. 3 depicts a single sensor 234 positioned adjacent each filter, multiple sensors 234 may be used instead. Using multiple sensors allows for redundancy in case one of the sensors fails. Additionally, using multiple sensors provides the ability to determine if there is more dust blockage on one side of the filter vs the other side of the filter, or on one side of the gantry vs the other side of the gantry. Using multiple sensors also allows for the cross-calibration of the sensors.

FIG. 4 depicts an example filter cleaning device 326 including a vacuum 402. The example vacuum includes a suction inlet 404 positioned adjacent the filter 302. The suction inlet 404 may be substantially the width of the filter 302 such that one pass of the vacuum 402 is able to clean the entire width of the filter 302. The example vacuum 402 is operated via one or more motor 406 or actuator. The motor 406 drives the vacuum along a set of tracks 408. The motor 406 may be coupled with any suitable device for moving the vacuum 402 along the set of tracks 408, including but not limited to a drive gear, a drive belt or chain, one or more wheels, and/or any combination thereof. The set of tracks 408 is positioned on either side of the filter 302. The set of tracks 408 has a length approximately the length of the filter 302. The example vacuum 420 includes an outlet 410 coupled to a collection bin 412 via a flexible hose 414. The example flexible hose has a length such that the vacuum 402 can travel to the furthest end of the set of tracks 408 and remain coupled to the collection bin 412 via the flexible hose 414.

During operation, the motor 406 may move the vacuum 402 from a starting position 416 (e.g., adjacent a top of the filter 302) to a second position 418 (e.g., adjacent a bottom of the filter 302) and then return to the starting position 416. During movement of the vacuum 402, the suction inlet 404 is adjacent to the filter 302 and is in operation to remove dirt from the surface of the filter 302 and collect the dirt in the collection bin 412. As such, the dirt is removed from the surface of the filter 302 and contained. In some examples, the collection bin may be instead integrated with the vacuum 402 such that the collection bin 412 moves with the vacuum and the flexible hose 414 is not needed. During regular preventative maintenance, a technician may empty the collection bin 412 and check the vacuum 402 and motor 406 for any repairs or replacement that may be needed. In some such example, an error message may be provided to a user via the display if the vacuum 402 or motor 406 become inoperable, and maintenance service may be scheduled.

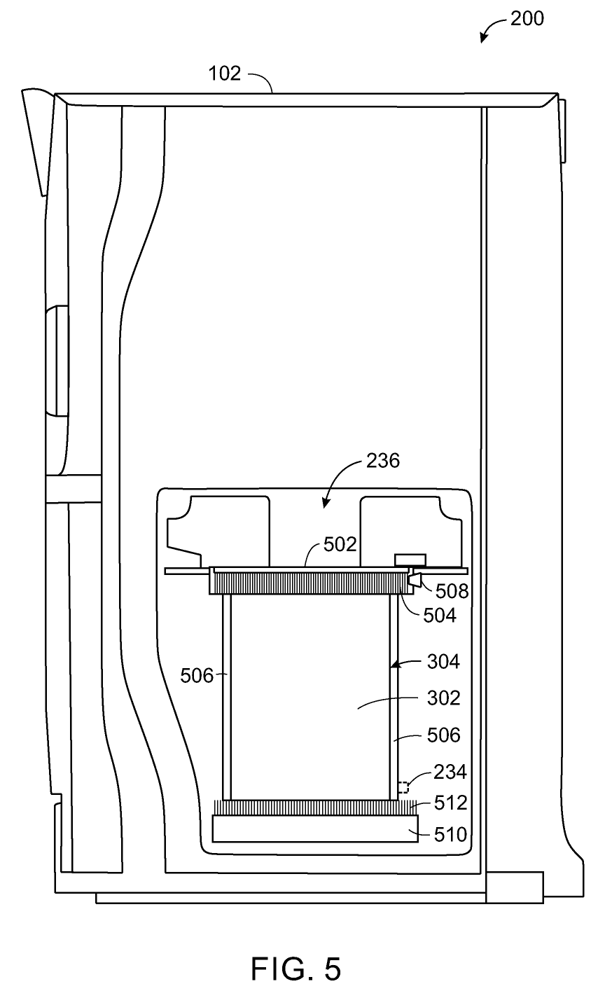

FIG. 5 depicts an example filter cleaning device 326 including a brush 502. The example brushy may include a plurality of soft bristles 504 (e.g., nylon bristles) that can remove dust and dirt from the surface of the filter 302 without damaging the filter 302. The bristles 504 may be any length sufficient to clean the surface of the filter 302. Additionally or alternatively, the example brush 502 may include adhesive and/or silicone components to trap dust and dirt. The example brush 502 may have a width approximately equal to the filter 302 to allow the brush 502 to clean the surface of the filter 302 with a single pass. The example brush 502 is coupled to a set of tracks 506 along which the brush 502 is moved. The set of tracks 506 may be approximately equal to the length of the filter 302. A motor 508 or actuator may be coupled to the brush 502 and operative to move the brush 502 along the set of tracks 506. The motor 508 may be coupled with any suitable device for moving the brush 502 along the set of tracks 506, including but not limited to a drive gear, a drive belt or chain, one or more wheels, and/or any combination thereof.

A collection bin 510 may be positioned adjacent a bottom edge of the filter 302 to collect dust and dirt removed from the surface of the filter 302 using the brush 502. In some examples, the collection bin 510 may include a comb 512 or other scraping device to facilitate removing trapped dirt from the bristles 504 of the brush 502. For example, the comb 512 may be positioned above an opening of the collection bin 510 such that the brush 502 passes over the comb 512 and the bristles 504 are moved through teeth of the comb 512 to clean the bristles 504. In some examples, the collection bin 510 includes an adhesive area to trap and/or remove dust and dirt from the bristles 504 of the brush 502. The example collection bin 510, brush 502, and/or comb 512 may be cleaned or replaced during regularly scheduled preventative maintenance. In some examples, if the motor 508 is inoperable, therefore, unable to perform cleaning tasks, an alert or error message may be provided to the operator or maintenance technician to schedule service.

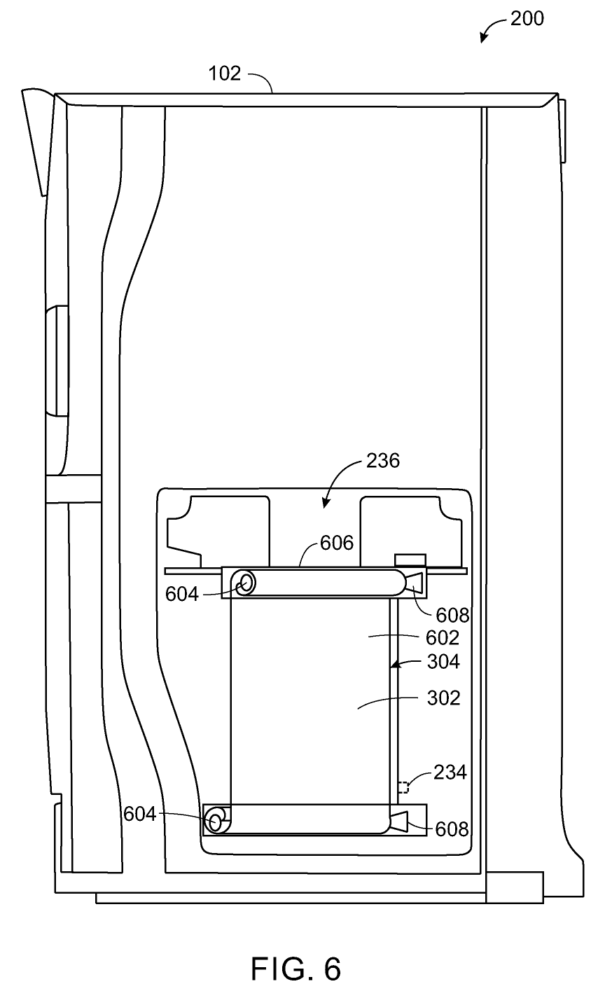

FIG. 6 depicts an example filter cleaning device 326 including a filter sheet 602 on a set of rotating rollers 604. The filter sheet 602 has a length at least twice the length of the filter 302. When the filter sheet 602 has too much dirt built up, the dirty portion of the filter sheet 602 is rolled onto a first roller of the set of rotating rollers 604 and a new section of the filter sheet 602 is simultaneously unrolled from a second roller of the set of rotating rollers 604. Each of the rollers 604 may be positioned in a housing 606. The housing 606 of the first roller 604 keeps the dust and dirt of the dirty section within the housing 606, and the housing 606 of the second roller 604 keeps dust and dirt from contaminating a clean section of the filter sheet 602. In some examples, each of the rollers 604 includes a respective motor 608 operative to rotate the corresponding roller 604. In such examples, the motors 608 may operate synchronously based on a single signal. Alternatively, a motor 608 is attached to one of the rollers (e.g., the first roller) and the other roller (e.g., the second roller) is free-spinning.

In some examples, the filter sheet 602 is a pre-filter and may be a thin material that allows the majority of dust and dirt to collect on the surface of the pre-filter sheet without significantly impacting the airflow through the filter 302. In some examples, the pre-filter may be a mesh-like material to trap the majority of the dirt without significantly affecting air flow. Alternatively, the filter sheet 602 is a HEPA filter capable of providing sufficient air filtration while also being flexible enough to be rolled around the set of rollers 604. In some examples, if the motor 608 is inoperable, therefore, unable to perform cleaning tasks, an alert or error message may be provided to the operator or maintenance technician to schedule service.

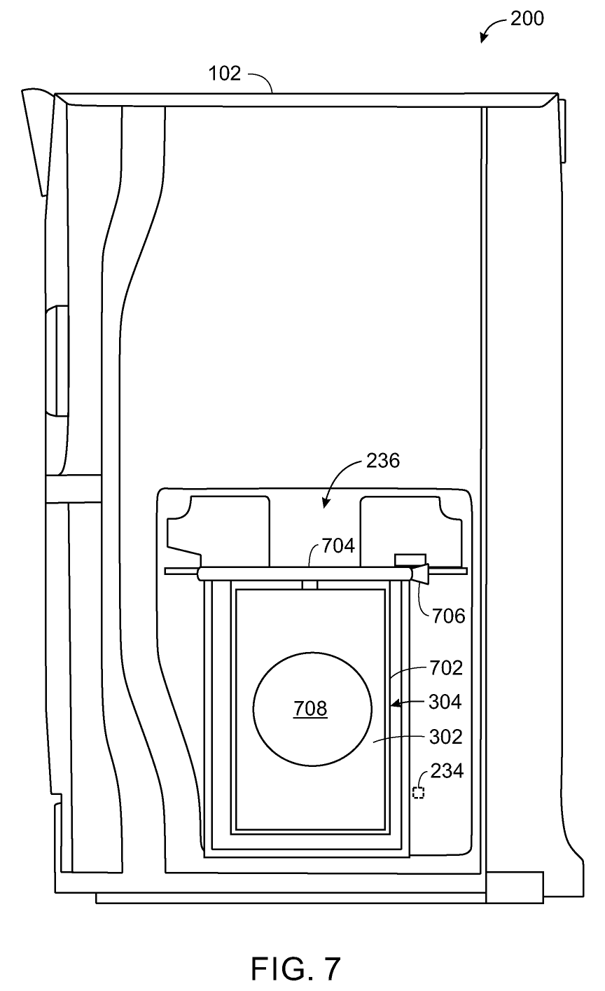

FIG. 7 depicts an example filter cleaning device 326 including and adhesive sheet 702 positioned over a roller 704. The example adhesive sheet may have a height and width corresponding to the height and width of the filter 302. To clean the filter 302, the adhesive sheet 702 may be unrolled over the filter 302. In some examples, the adhesive sheet is unrolled by moving the roller from a first end (e.g., a bottom end) of the filter 302 to a second end (e.g., a top end) of the filter. A motor 706 may facilitate movement of the roller (e.g., along a track). In some examples, the adhesive sheet 702 extends over the edges of the filter 302 to secure the adhesive sheet 702 in place as the filter 302 is cleaned. In some examples, the adhesive sheet 702 is sufficient to clean the filter 302. After the surface of the filter 302 is cleaned, the motor 706 is activated to re-roll the adhesive sheet 702 on the roller 704 as the roller 704 is returned to an initial position at a first end of the filter 302.

In some examples, after the adhesive sheet 702 is unrolled over the filter 302, a blower or fan 708 (shown for clarity) on the opposite side of the filter (e.g., inside the imaging system housing or gantry housing) blows air through the filter 302 to push dirt off the surface of the filter 302 and onto the adhesive sheet 702. In such examples, the fan 708 runs for a period of time (e.g., 10 seconds) to facilitate removal of the dirt from the surface of the filter 302. In some such examples, a material of the adhesive sheet 702 may include vents or opening to direct airflow while also allowing the dust or dirt to be trapped. For example, the adhesive sheet 702 may include micro holes or mesh areas, operative to collect some dirt while allowing air blown into the adhesive sheet 702 from the fan 708 to pass through. In some examples, one end or edge of the adhesive sheet 702 is unsecured (e.g., not adhered to the surface adjacent the filter 302) to allow air to escape. After the surface of the filter 302 is cleaned, the motor 706 is activated to re-roll the adhesive sheet 702 on the roller 704 as the roller 704 is returned to an initial position at a first end of the filter 302.

FIG. 8 is a flowchart depicting an example method 800 for scheduling filter maintenance of an imaging system. The example method begins at step 802 by establishing a baseline pressure differential over or across the filter 302. The example baseline pressure differential can be determined using the sensor 324 as described in conjunction with FIG. 2. The sensor may provide a signal to the computing device 216, which then analyzes the signal and determines the baseline pressure differential. Preferably, the baseline pressure differential is determined after installation of a new filter 302. In such examples, a maintenance technician may provide input via a user interface of the imaging system that a new filter 302 is installed, and the sensor 234 measures a baseline pressure differential. The baseline pressure differential is used as a comparative value for determining when the filter 302 should be cleaned or replaced. In examples where the sensor 234 is measuring a different value, a baseline value may be determined similarly, such that future measurements may be compared to the baseline value.

The method continues at step 804 by monitoring the pressure differential across the filter using the sensor 234. As discussed in conjunction with FIG. 2, the sensor 234 may include one or more sensors. If multiple filters are included in the imaging system, each filter may include at least one sensor 234. While monitoring the pressure differential via the sensor 324, the computing device 216 determines at step 806 if the pressure differential is outside a first pre-determined range. For example, the computing device 216 may determine if the pressure differential is below a threshold set based on the baseline pressure differential. In some such examples, the threshold may be a percentage of the baseline pressure differential. That is, is if the measured pressure differential is reduced by a predefined percentage, the pressure differential is below the threshold. In other examples where the sensor is measuring a different value, the computing device 216 may determine when the measured value is out of range compared to the baseline value (e.g., exceeds a range or threshold). If the pressure differential is not below the first threshold or the measured value is not out of the first range, the method returns to step 904 for continued monitoring. In some examples, monitoring is continuous, while in other examples, monitoring is periodic (e.g., performed at a set time interval, such as hourly, daily, etc.). Additionally or alternatively, the computing device 216 may determine filter cleaning or replacement is needed based on data trends or predictive modeling from the measured pressure differential over a set period of time.

If the pressure differential is above the first threshold or the measured value is outside the first range, an alert is provided to the operator via the display 232 of the imaging system 200 in step 808. In some examples, the alert is a pop-up alert. Alternatively, the alert may be included in a list of tasks to be performed by the operator. Other alerts may be provided to a remote monitoring or scheduling system (e.g., a fleet-wide monitoring station remote from the imaging system 200), or may be provided to a maintenance technician.

The method continues in step 810 by scheduling filter maintenance. In some examples, scheduling filter maintenance includes determining a time, based on a schedule of the imaging system 200 (e.g., a schedule of imaging scans to be performed using the imaging system). In such examples, the filter maintenance is scheduled for a time when the imaging system is not being used for patient scans. Additionally, the scheduled filter maintenance may be automatically rescheduled if additional imaging scan time is needed. In some examples, filter maintenance is performed automatically during the scheduled time using, for example, a filter cleaning device 236 such as that described herein in conjunction with FIGS. 4-7. In some examples, scheduling filter maintenance includes adding a filter maintenance task to a future or existing regularly scheduled imaging system preventative maintenance appointment. In such examples, the computing device 216 may track the task to add the task to a list of tasks to be performed by a maintenance technician. In other examples, the filter maintenance scheduling also includes scheduling a service appointment with a technician based on the availability of a technician and/or the schedule of the imaging system. In some examples, scheduling filter maintenance includes prompting the user to select or schedule a time for filter maintenance or for the filter cleaning device 236 to run a clean filter task.

At step 812, after the filter maintenance is scheduled, the computing device determines if filter cleaning was successful. In examples where filter cleaning is performed using a filter cleaning device 236, this may be determined based on the measured pressure differential using the one or more sensors 234. If the pressure differential is no longer below the threshold or other measured value is no longer out of range, the computing device 216 may determine that filter cleaning was successful. Alternatively, if the filter cleaning is performed by a technician, the technician may provide confirmation via the operator console 220 that the filter 302 was cleaned or replaced. If filter maintenance was successful, an alert is provided to the operator via the display 232 that the filter cleaning was successful in step 814. If the filter was replaced (as indicated by a technician), the method may return to step 802 to establish a new baseline pressure differential. If filter cleaning was not successful, an alert may be provided to the user that includes a prompt to schedule a maintenance appointment (or an additional maintenance appointment) in step 816. In such examples, the filter cleaning may not have been successful because one or more components of a filter cleaning device 236 is not operation. In examples where the operator is prompted to schedule a service appointment, the computing device 216 may provide appointment options based on technician availability and/or the imaging system schedule in step 818. Alternatively, the method may automatically schedule an appointment with a maintenance technician or an additional appointment with a maintenance technician. The method 800 is complete.

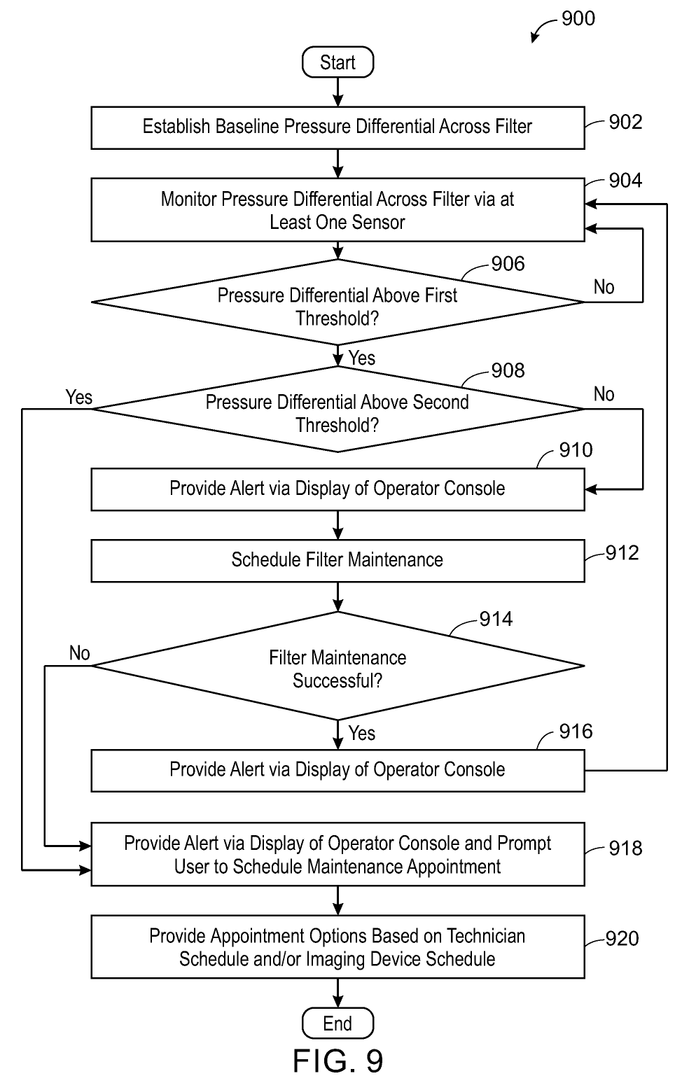

FIG. 9 is a flowchart depicting an example method 900 for scheduling filter maintenance of an imaging system. The example method begins at step 902 by establishing a baseline pressure differential over or across the filter 302. The example baseline pressure differential can be determined using the sensor 324 as described in conjunction with FIG. 2. The sensor may provide a signal to the computing device 216, which then analyzes the signal and determines the baseline pressure differential. Preferably, the baseline pressure differential is determined after installation of a new filter 302. In such examples, a maintenance technician may provide input via a user interface of the imaging system that a new filter 302 is installed, and the sensor 234 measures a baseline pressure differential. The baseline pressure differential is used as a comparative value for determining when the filter 302 should be cleaned or replaced. In examples where the sensor 234 is measuring a different value, a baseline value may be determined similarly, such that future measurements may be compared to the baseline value.

The method continues at step 904 by monitoring the pressure differential across the filter using the sensor 234. As discussed in conjunction with FIG. 2, the sensor 234 may include one or more sensors. If multiple filters are included in the imaging system, each filter may include at least one sensor 234. While monitoring the pressure differential via the sensor 324, the computing device 216 determines at step 906 if the pressure differential is outside a first pre-determined range. For example, the computing device 216 may determine if the pressure differential is below a first threshold set based on the baseline pressure differential. In some such examples, the first threshold may be a percentage of the baseline pressure differential. That is, is if the measured pressure differential is increased by a predefined percentage, the pressure differential is below the first threshold. In other examples where the sensor is measuring a different value, the computing device 216 may determine when the measured value is out of the first range compared to the baseline value (e.g., exceeds a range or threshold). If the pressure differential is not below the first threshold or the measured value is not out of range, the method returns to step 804 for continued monitoring. In some examples, monitoring is continuous, while in other examples, monitoring is periodic (e.g., performed at a set time interval, such as hourly, daily, etc.). Additionally or alternatively, the computing device 216 may determine filter cleaning or replacement is needed based on data trends or predictive modeling from the measured pressure differential over a set period of time.

If the pressure differential is above the first threshold or the measured value is out of the first range, the method continues to step 908 and determines if the pressure differential is outside a second pre-determined range. For example, the computing device 216 may determine if the pressure differential is below a second threshold set based on the baseline pressure differential. In some such examples, the second threshold may be a percentage of the baseline pressure differential. That is, is if the measured pressure differential is reduced by a second predefined percentage, greater than the percentage for the first threshold, the pressure differential is below the second threshold. In other examples where the sensor is measuring a different value, the computing device 216 may determine when the measured value is out of a second range compared to the baseline value (e.g., exceeds a range or threshold). Additionally or alternatively, the computing device 216 may determine filter cleaning or replacement is needed based on data trends or predictive modeling from the measured pressure differential over a set period of time.

If the pressure differential is not below the second threshold or the measured value is not out of the second range, an alert is provided to the operator via the display 232 of the imaging system 200 in step 910. In some examples, the alert is a pop-up alert. Alternatively, the alert may be included in a list of tasks to be performed by the operator. Other alerts may be provided to a remote monitoring or scheduling system (e.g., a fleet-wide monitoring station remote from the imaging system 200), or may be provided to a maintenance technician.

The method continues in step 912 by scheduling filter maintenance. In some examples, scheduling filter maintenance includes determining a time, based on a schedule of the imaging system 200 (e.g., a schedule of imaging scans to be performed using the imaging system). In such examples, the filter maintenance is scheduled for a time when the imaging system is not being used for patient scans. Additionally, the scheduled filter maintenance may be automatically rescheduled if additional imaging scan time is needed. In some examples, filter maintenance is performed automatically during the scheduled time using, for example, a filter cleaning device 236 such as that described herein in conjunction with FIGS. 4-7. In some examples, scheduling filter maintenance includes adding a filter maintenance task to a future or existing regularly scheduled imaging system preventative maintenance appointment. In such examples, the computing device 216 may track the task to add the task to a list of tasks to be performed by a maintenance technician. In other examples, the filter maintenance scheduling also includes scheduling a service appointment with a technician based on the availability of a technician and/or the schedule of the imaging system. In some examples, scheduling filter maintenance includes prompting the user to select or schedule a time for filter maintenance or for the filter cleaning device 236 to run a clean filter task.

At step 914, after the filter maintenance is scheduled, the computing device determines if filter cleaning was successful. In examples where filter cleaning is performed using a filter cleaning device 236, this may be determined based on the measured pressure differential using the one or more sensors 234. If the pressure differential is no longer above the threshold or other measured value is no longer out of range, the computing device 216 may determine that filter cleaning was successful. Alternatively, if the filter cleaning is performed by a technician, the technician may provide confirmation via the operator console 220 that the filter 302 was cleaned or replaced. If filter maintenance was successful, an alert is provided to the operator via the display 232 that the filter cleaning was successful in step 916. If the filter was replaced (as indicated by a technician), the method may return to step 902 to establish a new baseline pressure differential if the filter was replaced.

If, at step 908, the pressure differential is above the second threshold or other measured value is outside the second range, or if at step 914, filter cleaning was not successful, an alert may be provided to the user that includes a prompt to schedule a maintenance appointment (or an additional maintenance appointment) in step 918. In such examples, the filter cleaning may not have been successful because one or more components of a filter cleaning device 236 is not operation. In examples where the operator is prompted to schedule a service appointment, the computing device 216 may provide appointment options based on technician availability and/or the imaging system schedule in step 920. Alternatively, the method may automatically schedule an appointment with a maintenance technician or an additional appointment with a maintenance technician. The method 900 is complete.

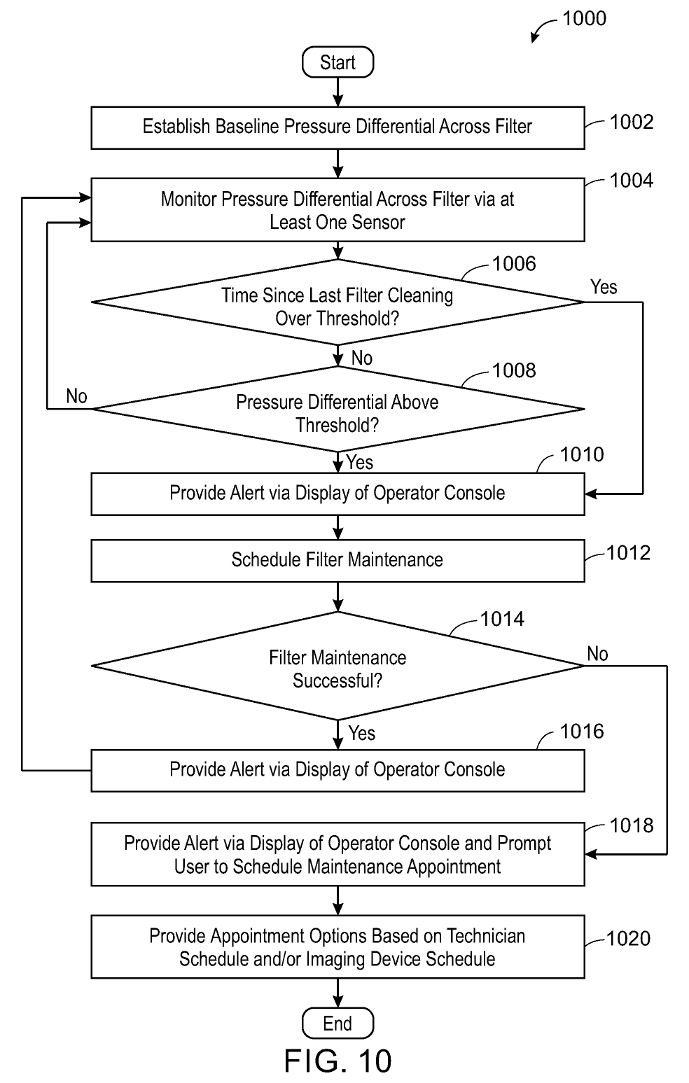

FIG. 10 is a flowchart depicting an example method 1000 for scheduling filter maintenance of an imaging system 200. The example method begins at step 1002 by establishing a baseline pressure differential over or across the filter 302. The example baseline pressure differential can be determined using the sensor 324 as described in conjunction with FIG. 2. The sensor may provide a signal to the computing device 216, which then analyzes the signal and determines the baseline pressure differential. Preferably, the baseline pressure differential is determined after installation of a new filter 302. In such examples, a maintenance technician may provide input via a user interface of the imaging system that a new filter 302 is installed, and the sensor 234 measures a baseline pressure differential. The baseline pressure differential is used as a comparative value for determining when the filter 302 should be cleaned or replaced. In examples where the sensor 234 is measuring a different value, a baseline value may be determined similarly, such that future measurements may be compared to the baseline value.

The method continues at step 1004 by monitoring the pressure differential across the filter using the sensor 234. As discussed in conjunction with FIG. 2, the sensor 234 may include one or more sensors. If multiple filters are included in the imaging imaging, each filter may include at least one sensor 234. In conjunction with monitoring the pressure differential via the sensor 324, the computing device may also monitor or track an amount of time that has passed since the last filter cleaning or replacement. If the computing device determines, in step 1006, that an amount of time since the last filter cleaning or replacement has exceeded a threshold amount of time, the method proceeds to step 1010. Until the amount of time since the last filter cleaning or replacement has exceeded a threshold amount of time, the method continues monitoring the pressure differential and proceeds to step 1008. At step 1008, the computing device 216 determines at step if the pressure differential is outside a first pre-determined range. For example, the computing device 216 may determine if the pressure differential is below a threshold set based on the baseline pressure differential. In some such examples, the threshold may be a percentage of the baseline pressure differential. That is, is if the measured pressure differential is reduced by a predefined percentage, the pressure differential is below the threshold. In other examples where the sensor is measuring a different value, the computing device 216 may determine when the measured value is out of range compared to the baseline value (e.g., exceeds a range or threshold). If the pressure differential is not below the first threshold or the measured value is not out of the first range, the method returns to step 1004 for continued monitoring. In some examples, monitoring is continuous, while in other examples, monitoring is periodic (e.g., performed at a set time interval, such as hourly, daily, etc.). Additionally or alternatively, the computing device 216 may determine filter cleaning or replacement is needed based on data trends or predictive modeling from the measured pressure differential over a set period of time.

If the pressure differential exceeds the threshold or if the measured value is outside of the range, an alert is provided to the operator via the display 232 of the imaging system 200 in step 1010. In some examples, the alert is a pop-up alert. Alternatively, the alert may be included in a list of tasks to be performed by the operator. Other alerts may be provided to a remote monitoring or scheduling system (e.g., a fleet-wide monitoring station remote from the imaging system 200), or may be provided to a maintenance technician.

The method continues in step 1012 by scheduling filter maintenance. In some examples, scheduling filter maintenance includes determining a time, based on a schedule of the imaging system 200 (e.g., a schedule of imaging scans to be performed using the imaging system). In such examples, the filter maintenance is scheduled for a time when the imaging system is not being used for patient scans. Additionally, the scheduled filter maintenance may be automatically rescheduled if additional imaging scan time is needed. In some examples, filter maintenance is performed automatically during the scheduled time using, for example, a filter cleaning device 236 such as that described herein in conjunction with FIGS. 4-7. In some examples, scheduling filter maintenance includes adding a filter maintenance task to a future or existing regularly scheduled imaging system preventative maintenance appointment. In such examples, the computing device 216 may track the task to add the task to a list of tasks to be performed by a maintenance technician. In other examples, the filter maintenance scheduling also includes scheduling a service appointment with a technician based on the availability of a technician and/or the schedule of the imaging system. In some examples, scheduling filter maintenance includes prompting the user to select or schedule a time for filter maintenance or for the filter cleaning device 236 to run a clean filter task.

At step 1014, after the filter maintenance is scheduled, the computing device determines if filter cleaning was successful. In examples where filter cleaning is performed using a filter cleaning device 236, this may be determined based on the measured pressure differential using the one or more sensors 234. If the pressure differential is no longer below the threshold or other measured value is no longer out of range, the computing device 216 may determine that filter cleaning was successful. Alternatively, if the filter cleaning is performed by a technician, the technician may provide confirmation via the operator console 220 that the filter 302 was cleaned or replaced. If filter maintenance was successful, an alert is provided to the operator via the display 232 that the filter cleaning was successful in step 1016. If the filter was replaced (as indicated by a technician), the method may return to step 1002 to establish a new baseline pressure differential. If filter cleaning was not successful, an alert may be provided to the user that includes a prompt to schedule a maintenance appointment (or an additional maintenance appointment) in step 1018. In such examples, the filter cleaning may not have been successful because one or more components of a filter cleaning device 236 is not operation. In examples where the operator is prompted to schedule a service appointment, the computing device 216 may provide appointment options based on technician availability and/or the imaging system schedule in step 1020. Alternatively, the method may automatically schedule an appointment with a maintenance technician or an additional appointment with a maintenance technician. The method 1000 is complete.

While multiple example methods for scheduling filter cleaning are described herein, it is understood that portions of the example methods may be removed, combined, rearranged, or added. For example, portions of the method 800 may be added to the methods 900 or 1000, portions of the method 900 may be added to the method 800 or 1000, and/or portions of the method 1000 may be added to method 800 or 900. Similarly, additional steps may be added to ore removed any of the example methods. Additionally, the steps of the methods as described herein may be rearranged and performed in a different order from that in the examples described.

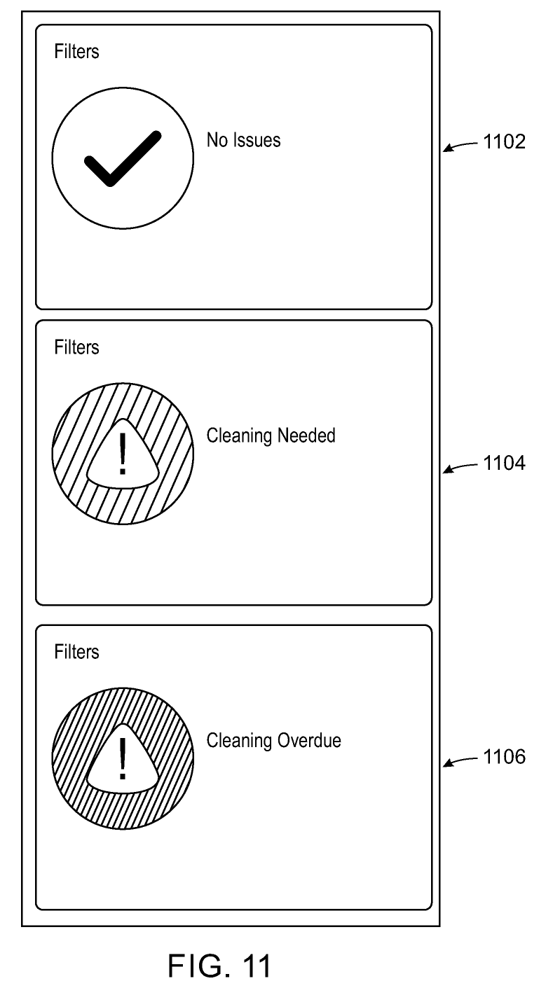

FIG. 11 depicts examples of indicators that may be provide to the operator via the display 232 regarding the stats of the filters 302. In some examples, the alerts may also provide an indication of which filters may need cleaning or replacement. The alerts may include a combination of text and icons, which may be color-coded based on the alert. Additionally, the alerts may include an interactive device (e.g., a button) the operator must select to acknowledge the alert. Indictor 1102 may be displayed to the operator when no issues are detected with the filter. That is, the pressure differential across the filter is not below the threshold and the filter does not need cleaned. Indicator 1104 may be displayed to the operator when the computing device 216 determines, based on the pressure differential measured by the sensors 234, that the filter needs cleaning. Indicator 1106 may be displayed to the operator if, for example, warnings to clean the filter are ignored for a period of time and the pressure differential still is below the threshold and/or the pressure differential has further decreased. Other indicators may be used in addition to the examples depicted in FIG. 11.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the claims. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method for scheduling maintenance of a filter of an imaging system, the method comprising:
detecting, via a sensor, a pressure differential across a filter;
providing a first alert to an operator, via a display of the imaging system, if the pressure differential across the filter is above a first threshold; and
scheduling filter maintenance based on the pressure differential being above the first threshold.

2. The method of claim 1, further comprising monitoring, via the sensor, the pressure differential across the filter.

3. The method of claim 1, further comprising establishing a baseline pressure differential, wherein the first threshold is a reduction of a measured value from the pressure differential.

4. The method of claim 1, wherein scheduling filter maintenance includes adding a filter maintenance task to an existing preventative maintenance appointment.

5. The method of claim 4, wherein the threshold comprises a first threshold and a second threshold, and the method further includes:
detecting, via the sensor, the pressure differential across the filter is above the second threshold;
providing a second alert to the operator that the pressure differential across the filter is above the second threshold; and
scheduling a maintenance appointment based on the pressure differential being above the second threshold.

6. The method of claim 5, wherein scheduling a maintenance appointment is based on an availability of a maintenance technician and a schedule of the imaging system.

7. The method of claim 1, wherein scheduling filter maintenance includes determining, based on a schedule of the imaging system, a time when the imaging system can run a filter cleaning task.

8. The method of claim 7, wherein running the filter cleaning tasks includes operating a filter cleaning device positioned adjacent the filter within the imaging system.

9. The method of claim 8, wherein the filter cleaning device removes dirt from the filter.

10. The method of claim 8, wherein the filter cleaning device moves the filter such that a clean portion of the filter is positioned over an air inlet of the imaging system.

11. The method of claim 8, further comprising providing a notification to the user that the filter has been successfully cleaned after running the filter cleaning task.

12. The method of claim 8, further comprising providing a notification to the that the filter cleaning task was not successful, and prompting the operator to schedule an appointment with a maintenance technician.

13. The method of claim 1, wherein scheduling filter maintenance includes prompting the operator to select a time for filter maintenance.

14. An imaging system, comprising:
a housing;
an inlet for air positioned within the housing;
a filter positioned adjacent to the inlet;
a display including a user interface;
one or more sensors adjacent to the filter, the one or more sensors to measure a pressure differential across the filter; and
a processor to:
determine the pressure differential is above a threshold;
provide an alert to an operator via the user interface of the display that the pressure differential is above the threshold; and
schedule filter maintenance based on pressure differential being above the threshold.

15. The imaging system of claim 14, wherein the processor is to schedule filter maintenance based on a schedule of the imaging system to determine a time when the imaging system can run a filter cleaning task.

16. The imaging system of claim 14, further including a filter cleaning device, wherein scheduling the filter maintenance includes scheduling a time to run the filter cleaning device.

17. The imaging system of claim 16, wherein the filter cleaning device includes a vacuum.

18. The imaging system of claim 16 wherein the filter cleaning device includes a brush.

19. The imaging system of claim 16, wherein the filter cleaning device includes a filter sheet having a length greater than the filter, wherein the filter sheet is positioned around one or more rollers, and wherein the rollers are rotated to move the filter sheet.

20. The imaging system of claim 14, wherein the processor is to further:
determine the pressure differential across the filter is above a second threshold;
provide a second alert to the operator that the pressure differential across the filter is above the second threshold; and
schedule a maintenance appointment based on the pressure differential being above the second threshold.
